(19) United States
(12) Patent Application Publication
(10) Pub. No.: US 2025/0347679 A1
(43) Pub. Date: Nov. 13, 2025

(54) SYSTEMS AND METHODS FOR OPTIMIZING AND SCREENING DRUG-RADIATION INTERACTIONS

(71) Applicant: Varian Medical Systems, Inc., Palo Alto, CA (US)

(72) Inventors: Angel Ku Villanueva, San Mateo, CA (US); Liang Tang, West Jordan, UT (US); Sophia Pfister, Redwood City, CA (US); Ricky Anupam Sharma, Lightwater (GB)

(21) Appl. No.: 18/663,047

(22) Filed: May 13, 2024

(51) Int. Cl. G01N 33/50; C12M 1/00; C12M 1/32; C12M 1/34

(52) U.S. Cl. CPC G01N 33/5011; C12M 23/12; C12M 29/06; C12M 41/46

(57) ABSTRACT

In some embodiments, a method of screening drug-radiation interactions includes determining a drug panel having a plurality of drugs to analyze; obtaining a cell line panel having one or more molecular parameters of interest and a plurality of cell lines; obtaining a cell line density and intrinsic radiotherapy response for each cell line in the cell line panel; determining a subset of drugs of the drug panel; determining initial radiation-dose-drug-concentration (RDDC) combinations for use with the subset of drugs of the drug panel; determining full concentration-response relationships for the cell lines of the cell line panel using the initial RDDC combinations; determining an RDDC combination subset from the initial RDDC combinations based on the full concentration-response relationships; and screening the cell lines of the cell line panel using the RDDC combination subset for each drug within the drug panel. Numerous other embodiments are provided.

### FIELD

The present application relates to drug and radiation therapies and more particularly to systems and methods for optimizing and screening drug-radiation interactions.

### BACKGROUND

Identifying promising drug-radiotherapy combinations can be expensive, time-consuming, and labor intensive. Current methods typically rely on hypothesis-driven testing of drug-radiotherapy combinations. Such methods limit the drug-radiotherapy space characterized and may miss numerous drug-radiotherapy combinations that reside outside the hypothesis employed. Further, such methods may be influenced by investigator bias.

Clonogenic survival assays are widely used in research and may be employed to examine the efficacy of drug-radiotherapy combinations. Such assays rely on the ability of a cell to divide and form a colony of a desired cell size (e.g., 50 cells) before and after drug-radiation therapy. While popular for studying drug-radiotherapy combinations, clonogenic survival assays are time-consuming, subjective, and labor-intensive, thus limiting the drug-radiotherapy space which can be efficiently explored.

Therefore, there is a need for improved methods and apparatus for optimizing and screening drug-radiation interactions.

### SUMMARY

In some embodiments, a method of screening drug-radiation interactions includes determining a drug panel having a plurality of drugs to analyze; obtaining a cell line panel having one or more molecular parameters of interest and a plurality of cell lines; obtaining a cell line density and intrinsic radiotherapy response for each cell line in the cell line panel; determining a subset of drugs of the drug panel; determining initial radiation-dose-drug-concentration (RDDC) combinations for use with the subset of drugs of the drug panel; determining full concentration-response relationships for the cell lines of the cell line panel using the initial RDDC combinations; determining an RDDC combination subset from the initial RDDC combinations based on the full concentration-response relationships; and screening the cell lines of the cell line panel using the RDDC combination subset for each drug within the drug panel.

In some embodiments, a method of screening drug-radiation interactions includes determining a drug panel having a plurality of drugs to analyze; obtaining a cell line panel having one or more molecular parameters of interest and a plurality of cell lines; obtaining a cell line density and intrinsic radiotherapy response for each cell line in the cell line panel; determining a subset of drugs of the drug panel; determining initial radiation-dose-drug-concentration (RDDC) combinations for use with the subset of drugs of the drug panel; employing correlation analysis to determine a single drug concentration from the initial RDDC combinations based on how the cell lines respond to the initial RDDC combinations; and screening the cell lines of the cell line panel using the single drug concentration for each drug within the drug panel.

In some embodiments, a system for screening drug-radiation interactions includes a dispenser system configured to dispense cells into cell lines of a cell line panel and drug concentrations into cell lines of the cell line panel; a radiation device configured to deliver radiation doses to the cell lines of the cell line panel; an incubator configured to incubate cell lines of the cell line panel; a measurement device configured to analyze the cell lines of the cell line panel; a processor coupled to the measurement device; and a memory coupled to the processor. The memory includes computer program instructions that, when executed by the processor, cause the processor to: obtain a list of drugs in a drug panel to analyze; obtain information related to a cell line panel having one or more molecular parameters of interest and a plurality of cell lines; obtain information related to a cell line density and intrinsic radiotherapy response for each cell line in the cell line panel; determine a subset of drugs of the drug panel; determine initial radiation-dose-drug-concentration (RDDC) combinations for use with the subset of drugs of the drug panel; determine full concentration-response relationships for the cell lines of the cell line panel using the initial RDDC combinations; determine an RDDC combination subset from the initial RDDC combinations based on the full concentration-response relationships; and screen the cell lines of the cell line panel using the RDDC combination subset for each drug within the drug panel.

In some embodiments, a system for screening drug-radiation interactions includes a dispenser system configured to dispense cells into cell lines of a cell line panel and drug concentrations into cell lines of the cell line panel; a radiation device configured to deliver radiation doses to the cell lines of the cell line panel; an incubator configured to incubate cell lines of the cell line panel; a measurement device configured to analyze the cell lines of the cell line panel; a processor coupled to the measurement device; and a memory coupled to the processor. The memory includes computer program instructions that, when executed by the processor, cause the processor to: obtain a list of drugs in a drug panel to analyze; obtain information related to a cell line panel having one or more molecular parameters of interest and a plurality of cell lines; obtain information related to a cell line density and intrinsic radiotherapy response for each cell line in the cell line panel; determine a subset of drugs of the drug panel; determine initial radiation-dose-drug-concentration (RDDC) combinations for use with the subset of drugs of the drug panel; employ correlation analysis to determine a single drug concentration from the initial RDDC combinations based on how the cell lines respond to the initial RDDC combinations; and screen the cell lines of the cell line panel using the single drug concentration for each drug within the drug panel.

Other features and aspects of the present invention will become more fully apparent from the following detailed description, the appended claims, and the accompanying drawings.

### BRIEF DESCRIPTION OF THE DRAWINGS

FIG. 1A illustrates a drug-radiation interaction screening system in accordance with embodiments provided herein.

FIG. 1B illustrates a computer of FIG. 1A configured in accordance with one or more embodiments provided herein.

FIG. 2A illustrates a high-level flowchart of a method of screening drug-radiation interactions in accordance with embodiments provided herein.

FIG. 2B illustrates a flowchart of an example embodiment of the method of FIG. 2A in accordance with embodiments provided herein.

FIG. 3 illustrates a plot of relative proliferation of cells versus drug concentration with and without radiation applied and normalized by cell growth without the drug compound applied, in accordance with embodiments provided herein.

FIG. 4 illustrates a plot of change in AUC versus change in cell count at a particular drug concentration for a subset of drug compounds examined in accordance with embodiments provided herein.

FIG. 5 illustrates a graph of Z-score counts versus Z-score of change in cell counts for each drug compound in a drug panel in accordance with embodiments provided herein.

FIG. 6 illustrates an example heat map based on a large drug panel screen in accordance with embodiments provided herein.

FIG. 7 illustrates a flowchart of a method of screening drug-radiation interactions in accordance with embodiments provided herein.

FIG. 8 illustrates a flowchart of another method of screening drug-radiation interactions in accordance with embodiments provided herein.

### DETAILED DESCRIPTION

Embodiments provided herein include systems and methods for an efficient approach to identifying effective drug-radiotherapy combinations. In at least some embodiments, such systems and methods may include a cell line screening component, a drug-radiation combination screening component, and/or a testing and characterization component. As described further below, such systems and methods allow for efficient screening of drug-radiation combinations that may be examined and validated. In some embodiments, screening only a subset of drug concentrations with radiotherapy allows efficient scoring of drug-radiation interactions and greatly increases the chemical space that may be experimentally characterized. For example, a typical drug-radiation interaction study might examine 180 drugs across 14 cell lines using 5-10 different concentrations for each drug, requiring 12, 600-25, 200 drug-radiation interactions to be examined (assuming a single radiation dose is employed). As described below, in some embodiments provided herein, a single drug concentration may be determined for such screening, reducing the drug-radiation interactions to be examined to 2,520. In larger studies, at least an order of magnitude more drug-radiation interactions may be examined.

For a cell line screening component, embodiments provided herein may allow for rapid testing of parameters relevant to screening cell lines which, in turn, may allow for rapid and efficient definition of optimal cell line screening parameters. For example, parameters relevant to high throughput drug-radiotherapy screening may include genetic mutations, gene expressions, protein expressions, protein activity, cell densities, growth kinetics such as exponential growth phase, growth conditions such as growth media, temperature, duration, etc., and/or the like. Knowledge of such parameters may allow cells to be classified into subsets having different parameters of interest. Cell line parameters may be determined through one or more of experimental analysis (e.g., studying kinetics of growth, dynamic range, genetic sensitivity, etc.), mining publicly available datasets (e.g., to identify relevant gene mutations, to determine growth dynamics and/or conditions, etc.), or the like.

For a drug-radiation combination screening component, in some embodiments, a screening platform is provided that employs the optimized cell-line screening parameters to characterize interactions between radiotherapy and drug therapies (providing optimized drug-radiation combination screening parameters). For example, optimized cell-lines may be radiated with a standard dose of radiation to determine an intrinsic sensitivity of each cell line to radiation (e.g., radiation resistant or radiation sensitive). Additionally, an optimal set of drug concentrations for use during screening of the cell lines may be determined (e.g., empirically tested and benchmarked drug concentrations). In some embodiments, a reduced or minimum set of drug concentrations may be determined (e.g., using computational modelling as described further below).

Once the optimized cell line and drug-radiation screening parameters have been determined, these parameters may be employed within a testing and characterization component to identify effective drug-radiation combinations. For example, in some embodiments, a system of one or more dispensers, radiation devices, incubators, measurement devices, and/or processing and scoring applications may be employed to carry out drug-radiation treatments on cell lines and to analyze and score and/or otherwise characterize the results of such treatments.

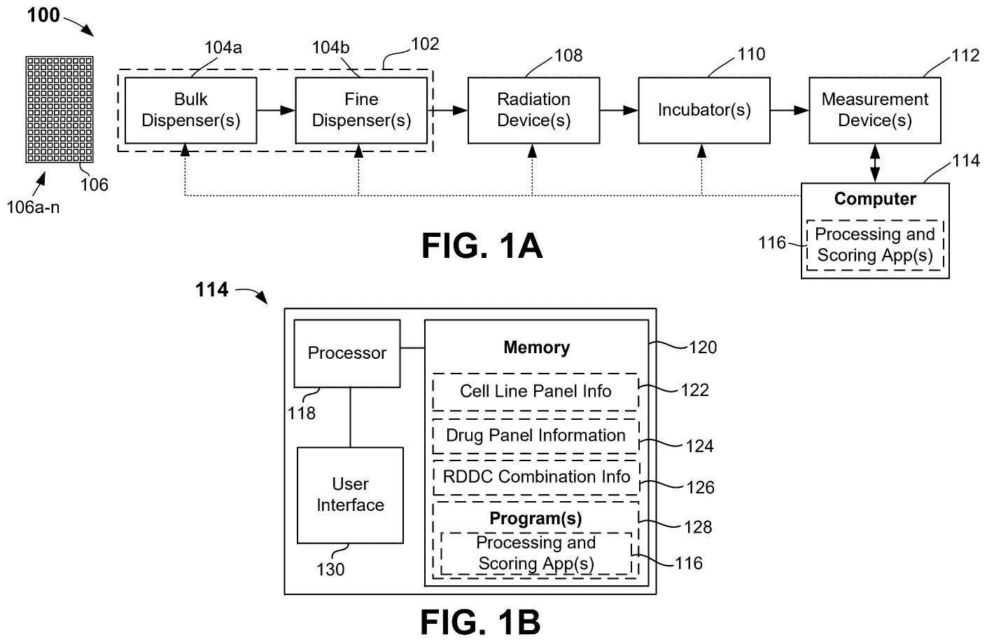

These and other embodiments of the invention are described below with reference to FIGS. 1A-8.

FIG. 1A illustrates a drug-radiation interaction screening system 100 in accordance with embodiments provided herein. With reference to FIG. 1A, system 100 includes a drug dispenser system 102 configured to dispense cells into cell lines of a cell line panel as well as drug concentrations into cell lines of the cell line panel. For example, dispenser system 102 may include a bulk dispenser 104a that provides cells to form cell lines 106a-n of a cell line panel 106 and a fine dispenser 104b for dispensing drug concentrations into the cell lines 106a-n. (For convenience, cell line panel 106 is shown within a single plate but, in some embodiments, may occupy several cell line plates in the case of a large cell line panel.) Alternatively, bulk dispenser 104a and fine dispenser 104b may dispense drug concentrations and/or fine dispenser 104b may dispense individual cells. In some embodiments, bulk dispenser 104a may dispense drug concentrations in the microliter to milliliter volume range, and fine dispenser 104b may dispense drugs concentrations in a nanoliter to microliter volume range. In some embodiments, the Multidrop™ Combi Reagent Dispenser available from Thermo Fischer Scientific, Inc. of Fair Lawn, NJ may be employed as the bulk dispenser 104a and the Echo 650 Liquid Handler available from Beckman Coulter, Inc. of Brea, CA may be employed as the fine dispenser 104b. Other dispenser types and/or configurations may be employed.

System 100 further includes a radiation device 108 configured to deliver ionizing radiation doses to cell lines 106a-n of cell line panel 106. In some embodiments, radiation device 108 may include one or more radiation sources and deliver any suitable form of radiation (e.g., conventional radiotherapy, FLASH radiotherapy, etc.). For example, in one or more embodiments, radiation doses may range from 0-20 Gy delivered at rates of 0.2 Gy/second to greater than 40 Gy/second and durations of less than a second to hours in length. Other radiation doses, rates, and/or durations may be employed. In some embodiments, radiation device 108 may include a MultiRad350 radiation system available from Precision X-ray Irradiation, Inc. of Madison, CT or a ProBeam, TrueBeam, and/or Edge x-ray system available from Varian Medical Systems, Inc. of Palo Alto, CA. Other radiation devices may be employed. Radiotherapy may include use of x-rays, electrons, photon, protons, or the like employing standard dosing, spatially fractionated radiation therapy (SFRT), stereotactic body radiation therapy (SBRT), ultra-high-dose-rate radiotherapy (e.g., FLASH radiotherapy), electron FLASH (eFLASH) radiotherapy, proton FLASH (pFLASH) radiotherapy, etc.

System 100 further includes an incubator 110 configured to incubate cell lines 106a-n of cell line panel 106 following drug delivery with drug dispensing system 102 and/or radiotherapy with radiation device 108. For example, incubator 110 may provide an environment that allows growth of the cells within cell lines 106a-n of cell line panel 106 following drug concentrations and radiotherapy. In some embodiments, incubator 110 may control temperature, humidity, and gas levels (e.g., carbon dioxide and oxygen levels) of cell lines 106a-n while maintaining a sterile environment for cell lines 106a-n. An example incubator may include the Thermo Scientific™ Heratherm™ oven available from Thermo Fischer Scientific, Inc. of Fair Lawn, NJ. Other incubator systems and/or multiple incubators may be employed. Example incubation environments include temperatures of 37° C.+/−5° C., 5-10% carbon dioxide, and 1-20% oxygen. Other incubation environments may be employed.

System 100 also includes a measurement device 112 configured to analyze cell lines 106a-n of cell line panel 106 following incubation within incubator 110 (as described further below). In some embodiments, measurement device 112 may include an optical measurement system configured to image, count and/or categorize cells of cell lines 106a-n following drug delivery (by dispenser system 102), radiation dosing (by radiation device 108), and/or incubation (within incubator 110). For example, measurement device 112 may determine how many cells within each cell line 106a-n are live following delivery of drug concentrations and/or radiotherapy. Other cell information may be determined such as images of cells, cells/well, etc. An example measurement device that may be suitable for use as measurement device 112 may include the ImageXpress® Confocal HT.ai imager available from Molecular Devices, LLC of San Jose, CA. Other measurement devices may be employed.

System 100 includes a computer 114 coupled to and configured to control measurement device 112 and receive and process measurement data from measurement device 112 (e.g., via one or more processing and scoring applications 116 (“apps”) described below). In some embodiments, computer 114 may also be coupled to and control operation of one or more of dispenser system 102, radiation device 108, and incubator 110.

FIG. 1B illustrates computer 114 of FIG. 1A in accordance with one or more embodiments. With reference to FIG. 1B, computer 114 includes a processor 118 coupled to a memory 120. Memory 120 may include information relevant to drug-radiation interaction screening such as optimized cell line panel information 122 (e.g., molecular parameters, cell line density, intrinsic radiotherapy response for each cell line in a cell line panel, etc.), drug panel information 124 (e.g., a list of drugs to be screened), radiation-dose drug-concentration (RDDC) information 126 (e.g., cell line radiation-dose drug-concentration (RDDC) combinations to test), or the like.

Memory 120 may also include one or more program(s) 128, such as computer executable instructions and/or code, for carrying out the methods described herein when executed by processor 118. As described further below, in one or more embodiments, program(s) 128 may include computer program instructions that, when executed by processor 118, cause processor 118 to obtain a list of drugs in a drug panel to analyze (e.g., drug panel information 124). For example, processor 118 may receive information via a user interface 130 (e.g., a touchscreen or other display, keyboard, microphone, etc.). As described in greater detail below, program(s) 128 may also include computer program instructions that, when executed by processor 118, cause processor 118 to obtain information related to a cell line panel having one or more molecular parameters of interest and a plurality of cell lines, as well as information related to a cell line density and intrinsic radiotherapy response for each cell line in the cell line panel (e.g., cell line panel information 122); determine a subset of drugs of the drug panel for optimizing drug concentrations (e.g., drug panel information 124); determine initial radiation-dose drug-concentration (RDDC) combinations for use with the subset of drugs of the drug panel (e.g., RDDC combination information 126); determine full concentration-response relationships for the cell lines of the cell line panel using the initial RDDC combinations; determine an RDDC combination subset from the initial RDDC combinations based on the full concentration-response relationships (e.g., RDDC combination information 126); and screen the cell lines of the cell line panel using the RDDC combination subset for each drug within the drug panel (e.g., via one or more processing and scoring apps 116).

In one or more embodiments, memory 120 may include computer program instructions that, when executed by processor 118, cause processor 118 to communicate with and control one or more of dispenser system 102 to dispense cell lines and/or drug concentrations into cell lines of a cell line panel, radiation device 108 to deliver radiation doses to cell lines of the cell line panel, incubator 110 to incubate cell lines of the cell line panel after drug delivery and/or radiotherapy, and measurement device 112 to measure one or more properties such as cell count, images of cells, cells/well, or the like of each cell line following incubation.

Processor 118 may be a computational resource such as, but not limited to, a microprocessor, a microcontroller, an embedded microcontroller, a digital signal processor (DSP), a field programmable gate array (FPGA) configured to perform as a microcontroller, or the like.

Memory 120 may be any suitable type of memory, such as, but not limited to, one or more of a volatile memory and/or a non-volatile memory. In one or more embodiments, memory 120 may be a non-transitory memory (e.g., a hard drive, a solid-state drive, a flash-drive, etc.). Memory 120 may have a plurality of instructions stored therein that, when executed by processor 118, cause processor 118 to perform various actions specified by one or more of the stored instructions. Memory 120 may include multiple memory units and/or types of memory. In some embodiments, all or a portion of memory 120 may be external to and/or remote from computer 114. Additionally, in some embodiments, multiple processors may be employed.

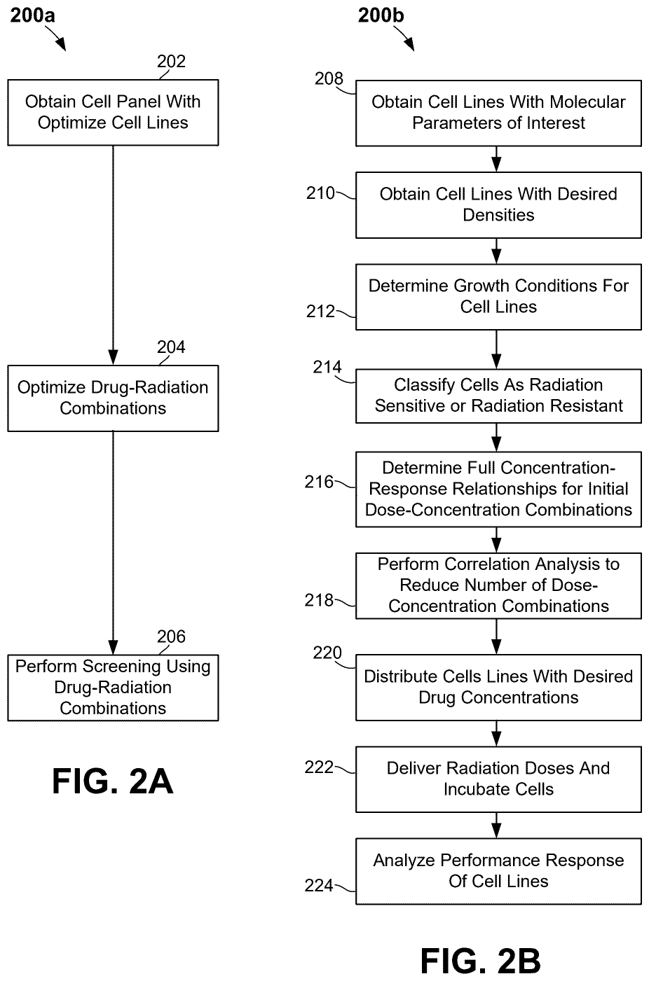

FIG. 2A illustrates a high-level flowchart of a method 200a of screening drug-radiation interactions in accordance with embodiments provided herein. At least portions of method 200a may be implemented within drug-radiation interaction screening system 100 of FIGS. 1A and 1B. Such an approach may be employed to screen (e.g., score) hundreds or even thousands of drug-radiotherapy interactions for drug panels including hundreds to thousands of compounds including, but not limited to, small molecule compounds, proteolysis targeting chimera (PROTAC) compounds, monoclonal antibodies (mAb), biologics (excluding mAb), enzyme inhibitors, or other compounds of interest.

With reference to FIG. 2A, in block 202, method 200a includes obtaining a cell line panel having optimized cell lines for drug-radiation interaction screening. In some embodiments, this may include determining the optimized screening conditions for molecularly defined cancer cell lines. As described further below, embodiments provided herein may allow for rapid testing of parameters relevant to screening cell lines which, in turn, may allow for rapid and efficient definition of optimal cell line screening parameters.

In block 204, method 200a includes optimizing drug-radiation combinations for drug-radiation interaction screening. In some embodiments, this may include employing the drug-interaction screening system 100 within the optimized cell-line screening parameters (block 202) to characterize interactions between radiotherapy and drug therapies (providing optimized drug-radiation combination screening parameters). For example, optimized cell-lines may be radiated with a standard dose of radiation to determine an intrinsic sensitivity of each cell line to radiation (e.g., radiation resistant or radiation sensitive). Additionally, an optimal set of drug concentrations for use during screening of the cell lines may be determined (e.g., empirically tested and benchmarked drug concentrations). In some embodiments, a reduced or minimum set of drug concentrations may be determined (e.g., using computational modelling as described further below).

In block 206, method 200a includes performing drug-radiation screening on the optimized cell line panel (block 202) using the optimized drug-radiation combinations (block 204) to identify effective drug-radiation combinations. For example, in some embodiments, drug-radiation interaction screening system 100 may be employed to carry out drug-radiation treatments on optimized cell lines (from block 202) using an optimized set of drug-concentration and radiation dose parameters (from block 204) and to analyze and score and/or otherwise characterize the results of such treatments (as described further below).

FIG. 2B illustrates a flowchart of an example embodiment of method 200a of screening drug-radiation interactions, referred to as method 200b, in accordance with embodiments provided herein. At least portions of method 200b may be implemented within drug-radiation interaction screening system 100 of FIGS. 1A and 1B.

With reference to FIG. 2B, in block 208, method 200b includes obtaining cell lines for a cell line panel with molecular parameters of interest for drug-radiation interaction screening (e.g., genetic, protein, or other molecular features of interest). In some embodiments, this may include creating a cell line panel that reflects the genetic heterogeneity of cancer for a cancer subtype of interest such as non-small-cell lung cancer, breast cancer, colon cancer, etc. Further, cell lines may have at least one of a genetic mutation, copy number variations, and a gene expression to be examined.

Method 200b also includes, in block 210, obtaining cell lines with desired densities and, in block 212, determining growth conditions for the cell lines. For example, block 210 may include determining the ideal plating (seeding) densities for each cell line of the cell line panel. In some embodiments, this may include an experimental framework in which dispenser system 102 may plate cells across several densities (e.g., 100 cells/well 500 cells/well, 1000 cells/well, etc.) and incubator 110 may incubate the plated cells. In block 212, image analysis (e.g., via measurement device 112) may be used to identify densities which facilitate exponential growth rates, and a timepoint (e.g., day, hours, minutes, etc.) at which each cell lines reaches a saturation point (e.g., 90-100%) confluency.

More generally, parameters relevant to high throughput drug-radiotherapy screening may include genetic mutations, gene expressions, cell densities, growth kinetics such as exponential growth phase, growth conditions such as growth media, temperature, duration, etc., and/or the like. Knowledge of such parameters may allow cells to be classified into subsets having different parameters of interest. Cell line parameters may be determined through one or more of experimental analysis (e.g., studying kinetics of growth, dynamic range, genetic sensitivity, etc.), mining publicly available datasets (e.g., to identify relevant gene mutations, to determine growth dynamics and/or conditions, etc.), or the like. Example cell seeding densities may range from about 250 to 2,000 cells per cell line, although other seeding densities may be employed. In some embodiments, assay length may range from about 7 to 10 days. Other assay lengths may be used. In some embodiments, dispenser system 102, incubator 110, and measurement device 112 of FIG. 1A may be employed to study cell line densities and/or growth conditions.

As a further example, to obtain a lung adenocarcinoma cell line panel, clinical data sets may be examined to identify a distribution of mutations that are commonly recurring in lung cancer. A cell line panel may then be designed that mimics the mutational distribution. For example, the KRAS mutation is present in up to 30% of lung cancer so a cell line panel may be developed that includes that amount of KRAS mutation as well as any other secondary and/or other mutations that drive lung cancer.

In some embodiments, computer 114 may be employed to computationally mine data sets and then computationally examine panels of genetically defined cancers and assess which are representative of what is observed in a clinic.

In some embodiments, quality control (QC) measures may be employed to verify cell line suitability. For example, cell lines may be examined for uniform growth across microplates and response to toxic drugs. A negative control may include use of drug solvent such as dimethylsulfoxide (DMSO) or water while a positive control may include a toxic substance such as staurosporine, another PKC inhibitor, another highly toxic compound, or an otherwise-relevant compound for a specific biological readout. In this manner, a dynamic range of the assays may be determined and verified as suitable (e.g., using Z′-factor). Further quality control measures may include reproducibility across technical replicates, or the like.

In block 214, method 200b includes classifying cells as radiation sensitive or radiation resistant. For example, cell lines may be treated with radiotherapy alone to determine their intrinsic response to radiotherapy (e.g., using radiation device 108). In some embodiments, this may include exposing cell lines to 2 Gy of radiation, incubating the cell lines, and determining a surviving cell density such as a relative proliferation of cell lines with and without radiation. For example, radiation device 108 may expose cell lines to radiation, incubator 110 may incubate the radiated cell lines, and measurement device 112 may facilitate determination of surviving cell density via computer 114.

In block 216, method 200b includes determining full concentration-response relationships for initial radiation-dose drug-concentration (RDDC) combinations. In some embodiments, this may include selecting a subset of drugs to examine and determining response curves for a range of drug concentrations and/or radiation doses. For example, testing a range of concentrations (e.g., 1 nanomolar to 100 micromolar) in combination with a range of radiotherapy doses (e.g., 0 Gy to 10 Gy) may be employed in some embodiments to determine full concentration-response curves for each drug in the subset.

During drug panel screening, testing 12 to 16 concentrations for each drug in combination with multiple radiation doses provides significant information regarding which drugs assist radiotherapy. However, for large drug panels such an approach is prohibitively time consuming. For example, for a 100-compound drug panel, testing 16 drug concentrations for each drug using 4 radiation doses requires 100×16×4=6400 combinations. For a 1000 compound drug panel, 64,000 combinations would need to be tested. As described below, in some embodiments, a single drug concentration (or a small number of drug concentrations) may be identified and used to test each drug compound in a large drug panel, significantly increasing the size of drug panels that may be screened efficiently.

In block 218, method 200b includes performing correlation analysis to reduce the number of RDDC combinations to be employed during drug panel screening. In some embodiments, correlation analysis based on area under curve (AUC) of full concentration-response curves or another metric may be used to identify a subset of drug concentrations (e.g., one or more drug concentrations) for use during drug panel screening. For example, in some embodiments, a single drug concentration may be employed when screening drug panels with thousands of different compounds versus more common approaches in which up to eight or more different drug concentrations may be used to screen drug panels of only 20 or 30 different compounds.

Once a subset of RDDC combinations has been developed (as described above in block 218), in block 220, method 200b includes distributing cell lines with desired drug concentrations. For example, for each drug compound in the drug panel to be screened, bulk dispenser 104a and/or fine dispenser 104b of drug-radiation interaction screening system 100 may be employed to distribute cell lines with the drug concentration(s) determined for the RDDC combination subset (block 218).

In block 222, method 200b includes delivering radiation doses and incubating cells lines. For example, radiation device 108 of drug-radiation interaction screening system 100 may be employed to deliver radiation to each cell line (formed in block 220) at doses determined by the RDDC combination subset (block 218). In some embodiments, this may include at least radiation doses of 0 Gy and 2 Gy, although more or different radiation doses may be employed (e.g., higher doses, standard radiation doses, high dose and low duration doses, etc.). Following radiation delivery (e.g., via radiation device 108), the cell lines may be incubated such as using incubator 110. Any suitable incubation conditions may be employed (e.g., such as the growth conditions determined in block 212).

In block 224, method 200b includes analyzing the performance response of the cell lines. Analysis may include measuring various properties of each cell line using measurement device 112 and computer 114 (e.g., cell density, Z-score counts versus Z-score of change in cell counts for each drug compound in a drug panel, etc.). In some embodiments, if only a single drug concentration is employed during screening of a cell line, then a single dose difference and ratio may be employed to quantify a difference between cell line response to the drug concentration with and without radiation. Further, response metrics may be Z-scored to compare across cell lines and used for hit identification.

### Overview of Initial Screening to Determine Single Drug Concentration for a Large Drug Panel

For a large drug panel having thousands of drug compounds, analyzing multiple radiation-dose, drug-concentration (RDDC) combinations for each drug is impractical. In accordance with embodiments provided herein, a subset of possible RDDC combinations including a plurality of different drug concentrations is examined and correlation analysis is employed to determine a single “optimized” drug concentration for screening of the full drug panel. The single, optimized drug concentration may be employed to screen each drug compound within the drug panel (e.g., using a control cell line panel exposed to the single drug concentration without radiation and a test cell line panel exposed to the single drug concentration with radiation for each drug compound in the drug panel).

As described, during examination of the subset of RDDC combinations, a plurality of drug concentrations may be tested for a subset of drug compounds within the drug panel. For example, in some embodiments, cell line panel responses to 5 or 6 drug concentrations for approximately 5 to 10 percent of the drugs within the drug panel may be examined with and without radiation (e.g., 0 Gy and 2 Gy). In other embodiments, one or more drug compounds that are not part of the drug panel may be employed to determine the optimal single drug concentration.

To examine the subset of RDDC combinations, for each drug compound in the subset of drugs, several concentrations of the drug compound are applied to cell line panels. For example, for a first drug compound A, a first cell line panel PIA may be exposed to a first concentration C1 of the first drug compound A, a second cell line panel P2A may be exposed to a second concentration C2 of the first drug compound A, a third cell line panel P3A may be exposed to a third concentration 3C of the first drug compound A, and the like. Similarly, a first cell line panel PIB may be exposed to the first concentration C1 of a second drug compound B, a second cell line panel P2B may be exposed to the second concentration C2 of the second drug compound B, a third cell line panel P3B may be exposed to the third concentration C3 of the second drug compound B, etc. For each drug compound concentration, two cell line panels may be employed. One cell line panel may be used as a control cell line panel (without radiation) and the other cell line panel may be used as the test cell line panel (with radiation). In this manner, a determination of the impact of the selected drug compound concentration on radiation sensitivity of cell lines may include comparing cell growth of a cell line panel with the drug compound concentration but no radiation to a cell line panel with the drug compound concentration plus radiation.

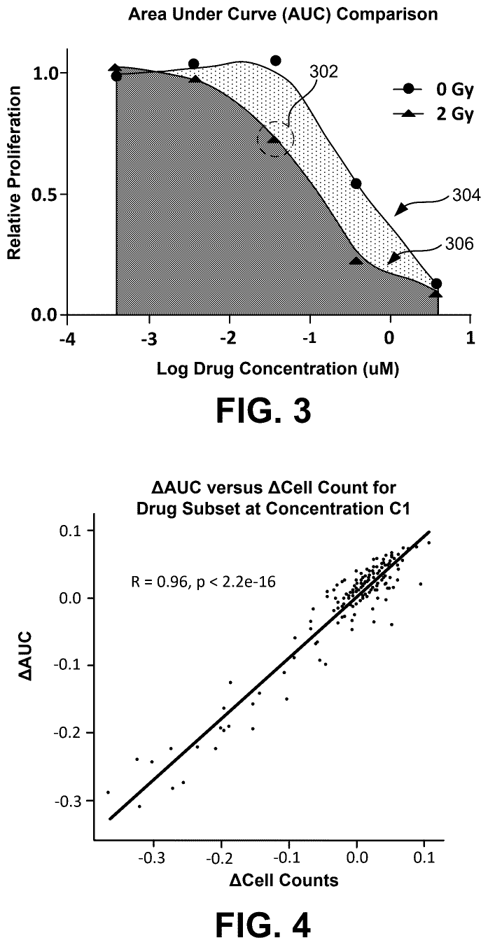

One metric that is widely used for examining cell line responses to a drug compound is half-maximal-inhibitory concentration (IC50). IC50 identifies the concentration of a drug compound at which cell growth is inhibited by 50%. However, the present inventors have observed that IC50 is highly dependent on the fitting model employed during drug-concentration radiation-dose combination screening. A single missed drug dose may create a large calculation error due to poor fitting. As such, in some embodiments provided herein, a change in area under the curve (AUC) of cell growth versus drug concentration is used to more accurately indicate and/or quantify drug-radiation response. For example, the change in AUC of cell growth versus drug concentration with and without radiation for a drug compound provides an estimate of how sensitive cells treated with the drug compound are to radiation. In some embodiments a regression technique (e.g., 4 parameter non-linear logistic regression or another regression technique) may be employed to fit a curve to cell growth, drug concentration data points as shown in FIG. 3 which illustrates a plot of relative proliferation of cells versus drug concentration with and without radiation applied, and normalized by cell growth without the drug compound applied, in accordance with embodiments provided herein. Specifically, in FIG. 3, the y-axis represents relative proliferation or a ratio of cell growth with the drug compound (with and without radiation) normalized by cell growth without the drug compound applied (e.g., with the solvent used to dissolve drug compounds such as water, DMSO, ethanol, etc.). Other relevant metrics may be employed such as survival rate.

The single optimized drug concentration for testing of the entire drug panel may be determined by identifying which drug concentration provides the largest decrease in cell growth when cells are exposed to the radiation dose employed. For example, in FIG. 3, the circled data point 302 may be selected for use as the single drug concentration (for use during screening of the overall drug panel). In practice, such AUC curves may be developed for each of the subset of drug compounds. Because the same drug concentrations (e.g., C1, C2, C3, etc.) are employed for each drug compound in the subset of drugs, the response of the cell lines to each drug concentration of each drug compound may be compared to identify the single drug concentration that produces the largest change in cell growth for the subset of drug compounds following radiation. For example, FIG. 4 illustrates a plot of change in AUC versus change in cell count at a particular drug concentration (Concentration C1) for the subset of drug compounds examined. As shown in FIG. 4, a linear fit and high R value of this data indicates that Concentration C1 is a viable candidate for use as the single, optimized drug concentration for the overall drug panel screening. This or another suitable correlation analysis technique may be employed to determine a single drug concentration (or a subset of drug concentrations in some embodiments) for use during screening of the cell lines. For example, change in IC50 or another metric may be plotted and fit to obtain a single drug concentration for use during overall drug panel screening.

In general, numerous concentrations along full concentration-response curves 304, 306 of FIG. 4 may be used to generate plots of change in AUC versus change in cell count (as in FIG. 4) and the concentrations providing a linear fit and high R value may be employed as suitable concentrations for use during overall drug panel screening. In some embodiments, two, three, or more concentrations providing linear fits and high R values may be employed within an RDDC combination subset for use during overall drug panel screening (e.g., as optimized drug concentrations).

### Overview of Drug Panel Screening

Cell lines of interest are identified for a cell line panel and the optimal plating conditions for the cell lines are determined (e.g., molecular parameters of interest, growth densities, growth conditions, etc.). For example, in some embodiments, cell lines may be derived from tumors of the same cancer type (e.g., non-small-cell lung cancer, breast cancer, colon cancer, etc.) and cell lines may represent different genomic features (e.g., different mutations, copy number variations, expression profiles, etc.). Drug compounds to be analyzed are identified (as a drug panel) and a single, optimized drug concentration may be determined (e.g., via correlation analysis as described above). (As stated, in some embodiments, more than one optimized drug concentration may be employed.) A separate cell line panel is established for each drug-concentration radiation dose combination to be tested within the drug panel. Thus, for a 4000-compound drug panel to be tested at radiation doses of 0 Gy and 2 Gy, 8000 technical replicate cell line panels are plated. For such a large drug panel study, the use of multiple drug concentrations and multiple radiation doses would be prohibitively time consuming.

Thus, use of a single drug concentration across a drug panel allows for efficient screening of large drug panels. That is, the approach of using a single drug concentration allows significantly more drug-radiation combinations to be analyzed than could be performed if a conventional approach were used in which many (e.g., 10 or more) drug concentrations are tested for each drug compound.

### EXAMPLE

In some embodiments, cells are plated at a desired density and incubated for a first incubation period (e.g., 24 hours). Example cell densities range from 50-2500 cells/well. Cells are plated for each cell line in a cell line panel and two identical cell line panels are established for each drug-concentration radiation-dose combination to be tested (e.g., a control cell line panel and test cell line panel for each drug concentration of each drug compound to be tested). Other incubation periods may be used.

After the first incubation period, for each drug compound to be tested, a single drug concentration (e.g., the single drug concentration identified by correlation analysis) is added to the cell lines of a control cell line panel associated with the drug compound (a cell line panel that will not be exposed to radiation) and to the cell lines of a test cell line panel associated with the drug compound (a cell panel that will be exposed to radiation). The cell line panels are then incubated for a second incubation period. In some embodiments, the second incubation period may be about 6 hours. Longer or shorter incubation periods may be employed.

After the second incubation period, for each drug compound to be tested, the test cell line panel for the drug compound is irradiated. In some embodiments, this may include delivery of a 2 Gy standard radiation dose. In general, any suitable radiation method may be employed such as x-ray, electron, photon, proton, etc., radiation employing standard dosing, SFRT, SBRT, ultra-high-dose-rate radiotherapy (e.g., eFLASH radiotherapy or pFLASH radiotherapy), etc. In other embodiments, cell lines may be irradiated and incubated prior to introducing drug compounds. In yet other embodiments, radiation dosing and drug delivery may be performed immediately after each other in either order without an intervening incubation period.

After radiation dosing, each cell line is incubated for a third incubation period until growth rates saturate or are near saturation (e.g., as determined by analyzing the cell lines). In some embodiments, the third incubation period may be between 3 and 7 days. Longer or shorter incubation periods may be employed.

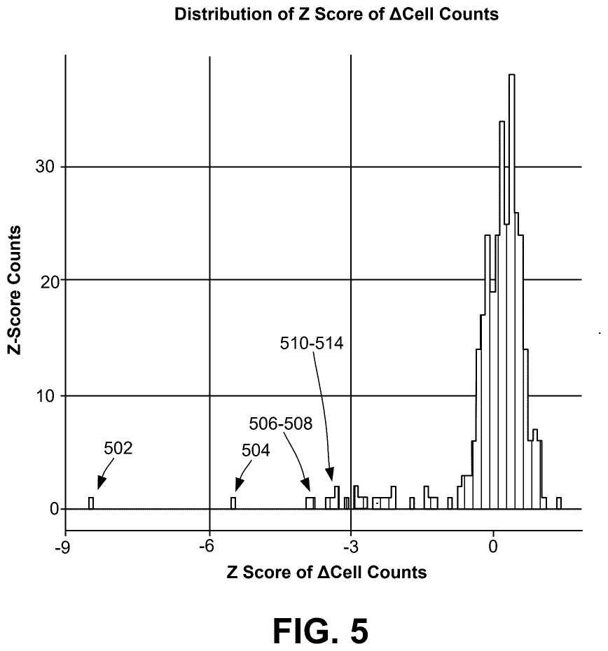

Following the third incubation period, the cell lines may be examined by comparing the test cell line panel to the control cell line panel for each drug compound in the drug panel. For example, FIG. 5 illustrates a graph of Z-score counts versus Z-score of change in cell counts for each drug compound in a drug panel. That is, the y-axis indicates how many drugs had a particular Z-score. As shown in FIG. 5, the majority of drugs tested had Z-scores near zero, indicating little impact of radiation on the cell lines treated with these drug compounds. For these drug compounds, the numbers of cells present in the cell lines with and without radiation were similar.

Drug compounds with larger Z-scores, such as indicated by arrows 502, 504, 506, 508, 510, 512, and 514 as well as others, exhibited a large difference in cell count with and without radiation and might be candidates for further study. For example, additional drug concentrations and/or radiation doses may be screened for these drug compounds. In some embodiments, change in AUC and/or IC50 may be calculated for the identified drug compounds to determine their viability for use during radiation treatment. In one or more embodiments, processing and scoring app 116 may determine Z-score counts versus Z-score change in cell counts, identify drug compounds with negative Z-scores (e.g., Z-scores below a predetermined threshold), alert a user of these drug compounds (e.g., via user interface 130), recommend further investigation of these drug compounds, initiate further investigation of such drug compounds, and/or the like.

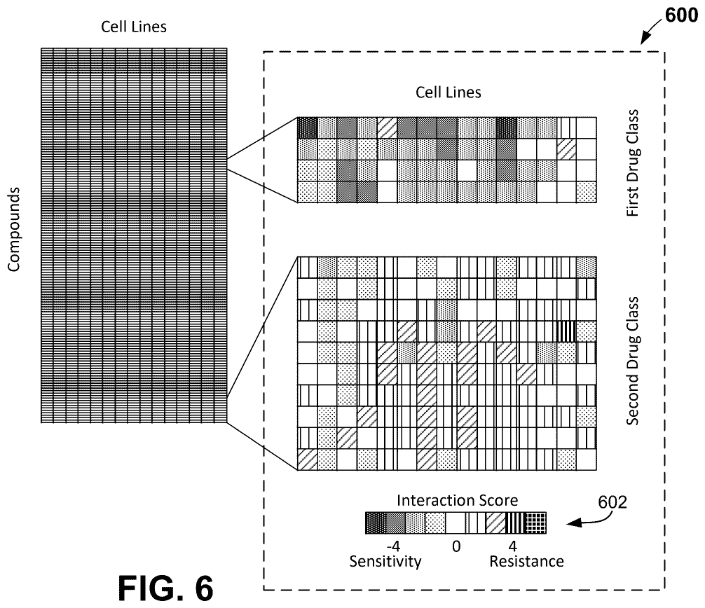

FIG. 6 illustrates an example heat map 600 based on a large drug panel screen in accordance with embodiments provided herein. For example, heat map 600 may employ an interaction score 602 based on Z-score of change in cell count with and without radiation as described above with regard to FIG. 5 and/or some other performance metric(s) such as strictly standardized mean difference (SSMD), B-score, R-score, quantile-based methods, or the like. As shown in FIG. 6 use of a single, optimized drug concentration during drug-radiation interaction screening allows for efficient identification of drug compounds that may be beneficial to radiotherapy treatments. In some embodiments, additional drug concentrations and/or radiation doses may be screened for drug compounds with interaction scores that identify the cell lines treated with these drug compounds as sensitive to radiation. In some embodiments, change in AUC and/or IC50 may be determined for drug compounds with interaction scores that identify the cell lines treated with these drug compounds as sensitive to radiation to determine their viability for use during radiation treatment.

In one or more embodiments, processing and scoring app 116 may automatically determine a heat map such as heat map 600 during drug-radiation interaction screening (e.g., with drug-radiation interaction screening system 100) and/or display a heat map using user interface 130.

### Quality Control

To validate testing, a quality control step may be performed to define a signal range between the lowest signal (e.g., no cell growth) and highest signal (e.g., normal cell growth) during analysis. Detection of the highest signal may include measuring cell growth after exposing cells to a solvent that does not inhibit cell growth (e.g., DMSO, ethanol, water, etc.) and detection of the lowest signal may include measuring cell growth after exposing cells to a toxic substance (e.g., staurosporine, another PKC inhibitor, another highly toxic compound, or an otherwise-relevant compound for a specific biological readout). In some embodiments, Z′-factor is determined based on this high and low signal range. For example, a Z-factor cutoff of 0.4 or another value may be employed to indicate that the screening is not performing optimally to distinguish a difference between high and low signals (e.g., below 0.4 indicating the screening is not performing optimally). Reproducibility quality checks may also be performed to validate reproducibility of results across cell line plates (e.g., by comparing cell growth rates, DMSO and staurosporine controls, signal-to-noise ratio, etc., for different cell line plates exposed to the same conditions).

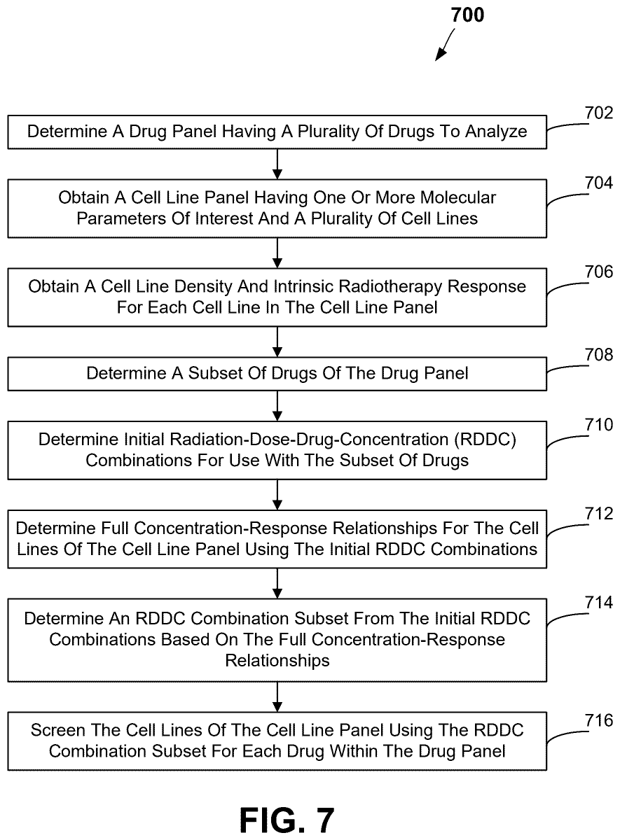

FIG. 7 is a flowchart of a method 700 of screening drug-radiation interactions in accordance with embodiments provided herein. In some implementations, one or more process blocks of FIG. 7 may be performed by drug-radiation interaction screening system 100 (FIG. 1A) using computer program instructions executable by processor 118 (FIG. 1B) such as program(s) 128 and/or processing and scoring app 116.

As shown in FIG. 7, process 700 may include determining a drug panel having a plurality of drugs to analyze (block 702). For example, computer 114 may input a list of drug compounds to analyze via user interface 130, obtain a list of drugs compounds to analyze from an external source (e.g., an external local or remote database), etc. In some embodiments, the drug panel may include clinically active drugs, novel chemical libraries without clinically active drugs, combinations of drugs, etc.

As also shown in FIG. 7, process 700 may include obtaining a cell line panel having one or more molecular parameters of interest and a plurality of cell lines (block 704). For example, cell lines may be obtained that have at least one of a genetic mutation, copy number variations, and a gene expression to be examined. In some embodiments, the cell line panel may have genetic heterogenicity for a cancer subtype (e.g., a lung adenocarcinoma cell line panel).

As further shown in FIG. 7, process 700 may include obtaining a cell line density and intrinsic radiotherapy response for each cell line in the cell line panel (block 706). In some embodiments, drug-radiation interaction screening system 100 (FIG. 1A) may be employed to determine cell line density and/or intrinsic radiotherapy response for each cell line in the cell line panel. For example, growth kinetics and growth conditions for the cell lines may be determined such as by plating cells across a plurality of cell densities (e.g., using dispenser system 102), identifying densities that facilitate exponential growth rates (e.g., using incubator 110, measurement device 112, and computer 114), and identifying a timepoint at which cell line growth rates saturate (e.g., using incubator 110, measurement device 112, and computer 114). Similarly, in some embodiments, radiation device 108, incubator 110, measurement device 112, and computer 114 may be employed to determine the intrinsic radiotherapy response of each cell line panel.

As also shown in FIG. 7, process 700 may include determining a subset of drugs of the drug panel (block 708). For example, in one or more embodiments, computer 114 and/or a user may select a subset of drugs of the drug panel. In some embodiments, 10% or fewer of the drugs of the drug panel may be selected (e.g., by a user or computer 114 such as automatically by processing and scoring app 116), while in other embodiments 5% or fewer of the drugs of the drug panel may be selected. Other numbers of drugs of the drug panel and/or drugs that are not within the drug panel may be selected for use as a subset.

As further shown in FIG. 7, process 700 may include determining initial radiation-dose-drug-concentration (RDDC) combinations for use with the subset of drugs of the drug panel (block 710). For example, program(s) 128, processing and scoring app 116, etc., may determine initial RDDC combinations for use with the subset of drugs of the drug panel, as described above. Initial RDDC combinations for use with the subset of drugs of the drug panel may include drug concentrations and radiation doses to apply to the cell lines of the cell line panels to be studied for the drug panel. In some embodiments, at least five drug concentrations may be selected (e.g., 5, 6, 7, 10, 15, or more different concentrations) at any desired concentration amount (e.g., nanomolar to micromolar). Further, in some embodiments, at least two radiation doses may be selected, such as 0 Gy and 2 Gy, although more and/or different numbers and amounts of radiation doses may be employed. Other numbers of drug concentrations may be employed.

As also shown in FIG. 7, process 700 may include determining full concentration-response relationships for the cell lines of the cell line panel using the initial RDDC combinations (block 712). For example, processor 118 via execution of computer program instructions (e.g., program(s) 128, processing and scoring app 116, etc.) may employ dispenser system 102 to plate cell lines with desired drug concentrations, radiation device 108 to radiate the plated cell lines, incubator 110 to incubate cell lines radiated and exposed to drug concentrations, and measurement device 112 to monitor cell growth. Processor 118 may then determine full concentration-response relationships for the cell lines of the cell line panel using the initial RDDC combinations.

In some embodiments, determining full concentration-response relationships for the cell lines of the cell line panel using the initial RDDC combinations may include, for each drug concentration of each drug of the subset of drugs and for each radiation dose to be examined: creating a duplicate cell line panel of the cell line panel (e.g., via dispenser system 102); exposing the duplicate cell line panel to the drug concentration and the radiation dose (e.g., via dispenser system 102 and radiation device 108); and measuring a cell count of each cell line of the duplicate cell line panel following exposure to the drug concentration and radiation dose (e.g., via measurement device 112). In some embodiments, processing and scoring app 116 may determine relative proliferation versus drug concentration and fit a curve to such data (e.g., as shown in FIG. 3).

As further shown in FIG. 7, process 700 may include determining an RDDC combination subset from the initial RDDC combinations based on the full concentration-response relationships (block 714). That is, the initial RDDC combination may include more drug concentrations and/or more radiation doses than would be practical for a large drug panel screen. As such, a reduced RDDC combination subset may be determined that employs fewer drug concentrations (e.g., 3 or fewer in some embodiments, and only one in some embodiments) and fewer radiation doses (e.g., a single non-zero radiation dose such as 2 Gy). For example, in some embodiments, processor 118 may execute computer program instructions (e.g., program(s) 128 and/or processing and scoring app 116) to determine an RDDC combination subset from the initial RDDC combinations based on the full concentration-response relationships, as described above.

In some embodiments, determining an RDDC combination subset from the initial RDDC combinations may include: for each drug of the subset of drugs, creating full concentration-response curves for the drug with and without radiation and computing a change in area under curve (AUC) between the full concentration-response curves for the drug with and without radiation; and employing change in AUC for each drug of the subset of drugs to determine one or more drug concentrations for the RDDC combination subset. For example, processor 118 and program(s) 128 and/or processing and scoring app 116 may generate full concentration-response curves for each drug to be tested with and without radiation and compute change in AUC based thereon as described previously. In some embodiments, change in AUC versus change in cell count may be plotted for all drugs in the drug subset and regression techniques may be employed to provide a linear fit to obtain an optimized single drug concentration to employ during screening of the entire drug panel. In some embodiments, processing and scoring app 116 may perform such linear fitting to obtain one or more optimized drug concentrations.

In further embodiments, determining an RDDC combination subset from the initial RDDC combinations may include: determining an initial set of drug concentrations (e.g., automatically, user-selected, etc.); determining how the cell lines respond to the initial set of drug concentrations and a range of radiation doses (e.g., via full concentration-response curves); employing correlation analysis (e.g., linear regression) to determine a subset of drug concentrations from the initial set of drug concentrations based on how the cell lines respond to the initial set of drug concentrations and the range of radiation doses; and determining the RDDC combination subset based on the subset of drug concentrations (as described above).

As also shown in FIG. 7, process 700 may include screening the cell lines of the cell line panel using the RDDC combination subset for each drug within the drug panel (block 716). For example, computer 114 using processor 118 and computer program instructions (e.g., program(s) 128 and/or processing and scoring app 116) may screen the cell lines of the cell line panel using the RDDC combination subset for each drug within the drug panel, as described above. In some embodiments, screening the cell lines of the cell line panel using the RDDC combination subset for each drug within the drug panel may include, for each drug of the drug panel and for each drug concentration and each radiation dose of the RDDC combination subset: creating a duplicate cell line panel of the cell line panel (e.g., via dispenser system 102); exposing the duplicate cell line panel to the drug concentration and the radiation dose (e.g., via dispenser system 102, radiation device 108, and incubator 110); and measuring a cell count of each cell line of the duplicate cell line panel following exposure to the drug concentration and radiation dose (e.g., via measurement device 112).

As shown in FIG. 6, in some embodiments, processor 118 executing computer program instructions (e.g., program(s) 128 and/or processing and scoring app 116) may generate a heat map based on a large drug panel screen. For example, the heat map may employ an interaction score based on Z-score of change in cell count with and without radiation as described above with regard to FIG. 5 and/or some other performance metric(s) such as SSMD, B-score, R-score, quantile-based methods, or the like. As shown in FIG. 6 use of a single, optimized drug concentration during drug-radiation interaction screening allows for efficient identification of drug compounds that may be beneficial to radiotherapy treatments. In some embodiments, additional drug concentrations and/or radiation doses may be screened for drug compounds with interaction scores that identify the cell lines treated with these drug compounds as sensitive to radiation. In some embodiments, change in AUC and/or IC50 may be determined for the identified drug combinations to determine their viability for use during radiation treatment.

Although FIG. 7 shows example blocks of process 700, in some implementations, process 700 may include additional blocks, fewer blocks, different blocks, or differently arranged blocks than those depicted in FIG. 7. Additionally, or alternatively, two or more of the blocks of process 700 may be performed in parallel.

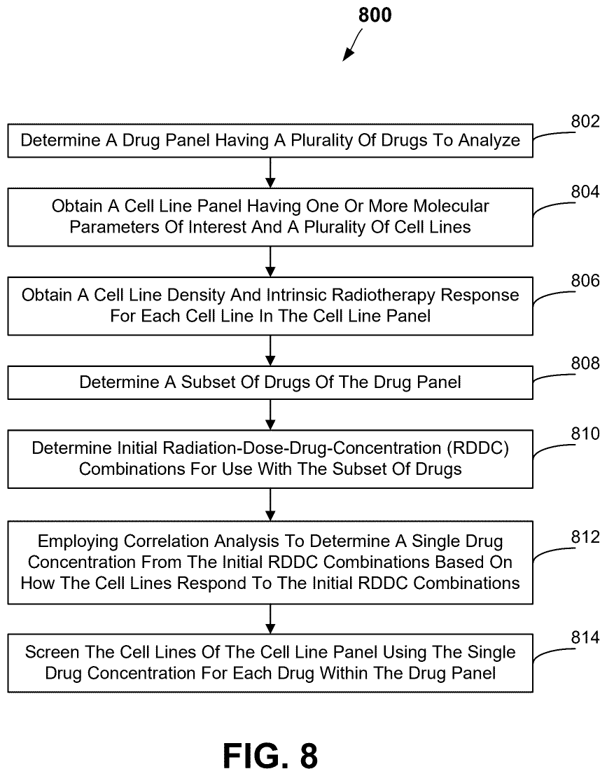

FIG. 8 is a flowchart of a method 800 of screening drug-radiation interactions in accordance with embodiments provided herein. In some implementations, one or more process blocks of FIG. 8 may be performed by drug-radiation interaction screening system 100 (FIG. 1A) using computer program instructions executable by processor 118 (FIG. 1B) such as program(s) 128 and/or processing and scoring app 116.

As shown in FIG. 8, process 800 may include determining a drug panel having a plurality of drugs to analyze (block 802). For example, processor 118 executing computer program instructions (e.g., program(s) 128 and/or processing and scoring app 116) may determine a drug panel having a plurality of drugs to analyze, as described above.

As also shown in FIG. 8, process 800 may include obtaining a cell line panel having one or more molecular parameters of interest and a plurality of cell lines (block 804). For example, processor 118 executing computer program instructions (e.g., program(s) 128 and/or processing and scoring app 116) may obtain a cell line panel having one or more molecular parameters of interest and a plurality of cell lines, as described above.

As further shown in FIG. 8, process 800 may include obtaining a cell line density and intrinsic radiotherapy response for each cell line in the cell line panel (block 806). For example, processor 118 executing computer program instructions (e.g., program(s) 128 and/or processing and scoring app 116) may obtain a cell line density and intrinsic radiotherapy response for each cell line in the cell line panel, as described above.

As also shown in FIG. 8, process 800 may include determining a subset of drugs of the drug panel (block 808). For example, processor 118 executing computer program instructions (e.g., program(s) 128 and/or processing and scoring app 116) may determine a subset of drugs of the drug panel, as described above.

As further shown in FIG. 8, process 800 may include determining initial RDDC combinations for use with the subset of drugs of the drug panel (block 810). For example, processor 118 executing computer program instructions (e.g., program(s) 128 and/or processing and scoring app 116) may determine RDDC combinations for use with the subset of drugs of the drug panel, as described above.

As also shown in FIG. 8, process 800 may include employing correlation analysis to determine a single drug concentration from the initial RDDC combinations based on how the cell lines respond to the initial RDDC combinations (block 812). For example, processor 118 executing computer program instructions (e.g., program(s) 128 and/or processing and scoring app 116) may determine full concentration-response relationships for the cell lines of the cell line panel using the initial RDDC combinations, as described above.

As also shown in FIG. 8, process 800 may include screening the cell lines of the cell line panel using the single drug concentration for each drug within the drug panel (block 814). For example, processor 118 executing computer program instructions (e.g., program(s) 128 and/or processing and scoring app 116) may screen the cell lines of the cell line panel using the single drug concentration for each drug within the drug panel, as described above.

Although FIG. 8 shows example blocks of process 800, in some implementations, process 800 may include additional blocks, fewer blocks, different blocks, or differently arranged blocks than those depicted in FIG. 8. Additionally, or alternatively, two or more of the blocks of process 800 may be performed in parallel.

In some embodiments, once data is collected, data is processed and normalized. For each drug concentration and radiation dose combination and each cell line, performance may be assessed using screening metrics such as, for example, Z′-factor, signal-to-noise ratio (S/N), technical replicate correlation (e.g., Pearson's correlation coefficient), or the like. Cell lines that pass quality control may be further analyzed. For example, in some embodiments, when multiple drug concentrations are employed during screening, area-under-curve (AUC) may be derived for cell lines with and without radiation dosing.

In some embodiments, radiation doses ranging from 0-10 Gy may be employed. In other embodiments, short duration radiation doses of 20-50 Gy may be employed (e.g., with doses of less 1 second).

In one or more embodiments, change in AUC may be computed for each drug to be screened. A library of changes in AUC may be created (e.g., a database of change in AUC for each drug may be stored in memory 120).

Methods, apparatus, and systems described herein may allow for the rapid testing of several experimental parameters to define the optimal parameters for screening panels of cell lines such as cancer cell lines for drug-assisted radiotherapy applications. This may include optimizing the parameters for high throughput drug-radiotherapy screening (e.g., cell line panels of interest, growth conditions and kinetics, etc.). Further, a screening platform may be provided to take the optimized screening parameters and characterize the interactions between radiotherapy and drug therapies. This may include determination of a reduced-size set of drug concentrations, which are empirically tested and benchmarked. Additionally, a workflow is provided that allows for the screening of drug-radiotherapy combinations using a specific network of hardware (e.g., drug-radiation interaction screening system 100). In addition, software and code (e.g., program(s) 128 and/or processing and scoring app 116) may be provided to assess quality and reproducibility of each step in the workflow.

As stated, in some embodiments, radiotherapies may include but are not limited to photon, electron, and proton external beam radiation, as well as short duration-high dose radiation (e.g., FLASH).

Using an optimized subset of drug-radiation combinations allows for the efficient screening of radiosensitizers which can be followed up and validated. In some embodiments, only screening with a subset of drug concentrations with radiotherapy (e.g., only a single drug concentration in some embodiments) allows for very efficiently scoring of drug-radiotherapy interactions. This approach greatly increases the chemical space that can be characterized per experiment. For example, with a panel of 180 drugs and 14 cells, over 12,000 drug-radiotherapy interactions may be investigated. In some embodiments, over 110,000 drug-radiotherapy interactions across panels of cell lines and drugs may be investigated.

One or more embodiments provided herein may be used to test drug-radiotherapy combinations and screen novel chemical libraries without clinically active drugs. Further, in some embodiments, drug-drug interactions may be screened to characterize efficacy and toxicity. Embodiments provided herein may be used in further miniaturizing for 1536-well plates or implementing alternative assays (e.g., CTG or MIT assays). In addition, in some embodiments, patient-derived cell lines may be screened for precision medicine applications. For example, cell lines may be derived from patient samples and patient-derived cell models.

In some embodiments, memory 120 may include computer program instructions (e.g., program(s) 128 and/or processing and scoring app 116) that, when executed by processor 118, cause processor 118 to: for each drug within a drug panel and a drug concentration, employ measurement device 112 to measure cell count within each cell line of a cell line panel with and without radiation; compare Z-score of change in cell count with and without radiation for each drug within the drug panel; and identify each drug within the drug panel having a Z-score of change in cell count below a predetermined threshold (e.g., non-zero, less than zero, less than one standard deviation, less than two standard deviations, or some other predetermined threshold).

In some embodiments, memory 120 may include computer program instructions (e.g., program(s) 128 and/or processing and scoring app 116) that, when executed by processor 118, cause processor 118 to measure the cell line density of each cell line of a cell line panel by: employing dispenser system 102 to plate cells across a plurality of cell densities; employing incubator 110 to incubate the plated cells; employing measurement device 112 to identify densities that facilitate exponential growth rates; and identifying a timepoint at which cell line growth rate saturates.

In some embodiments, memory 120 may include computer program instructions (e.g., program(s) 128 and/or processing and scoring app 116) that, when executed by processor 118, cause processor 118 to measure the intrinsic radiotherapy response of each cell line of a cell line panel by: employing dispenser system 102 to create a duplicate cell line panel of the cell line panel; employing radiation device 108 to expose the duplicate cell line panel to a predetermined radiation dose; employing incubator 110 to incubate the duplicate cell line panel; and employing measurement device 112 to measure the cell count of each cell line of the duplicate cell line panel to determine the intrinsic radiotherapy response of the cell lines of the duplicate cell line panel.

In some embodiments, memory 120 may include computer program instructions (e.g., program(s) 128 and/or processing and scoring app 116) that, when executed by processor 118, cause processor 118 to determine full concentration-response relationships for the cell lines of a cell line panel using initial RDDC combinations by, for each drug concentration of each drug (of the subset of drugs of a drug panel) and each radiation dose to be examined, (1) employing dispenser system 102 to create a duplicate cell line panel of the cell line panel; (2) employing dispenser system 102 to expose the duplicate cell line panel to the drug concentration; (3) employing radiation device 108 to expose the duplicate cell line panel to the radiation dose; and (4) employing measurement device 112 to measure a cell count of each cell line of the duplicate cell line panel following exposure to the drug concentration and radiation dose.

### Non-Limiting Illustrative Embodiments

The following is a list of non-limiting embodiments of inventive concepts disclosed herein:

An illustrative method of screening drug-radiation interactions, comprising: determining a drug panel having a plurality of drugs to analyze; obtaining a cell line panel having one or more molecular parameters of interest and a plurality of cell lines; obtaining a cell line density and intrinsic radiotherapy response for each cell line in the cell line panel; determining a subset of drugs of the drug panel; determining initial radiation-dose-drug-concentration (RDDC) combinations for use with the subset of drugs of the drug panel; determining full concentration-response relationships for the cell lines of the cell line panel using the initial RDDC combinations; determining an RDDC combination subset from the initial RDDC combinations based on the full concentration-response relationships; and screening the cell lines of the cell line panel using the RDDC combination subset for each drug within the drug panel.

The illustrative method of any of the proceeding illustrative embodiments wherein obtaining a cell line panel having one or more molecular parameters of interest comprises obtaining cell lines having at least one of a genetic mutation, copy number variations, and a gene expression to be examined.

The illustrative method of any of the proceeding illustrative embodiments wherein the cell line panel comprises genetic heterogenicity for a cancer subtype.

The illustrative method of any of the proceeding illustrative embodiments wherein obtaining a cell line density for each cell line in the cell line panel includes at least one of determining growth kinetics and growth conditions for the cell line.

The illustrative method of any of the proceeding illustrative embodiments wherein determining a cell line density for each cell line comprises: plating cells across a plurality of cell densities; identifying densities that facilitate exponential growth rates; and identifying a timepoint at which cell line growth rate saturates.

The illustrative method of any of the proceeding illustrative embodiments wherein determining a subset of drugs of the drug panel comprises selecting 10% or fewer of the drugs of the drug panel.

The illustrative method of any of the proceeding illustrative embodiments wherein determining a subset of drugs of the drug panel comprises selecting 5% or fewer of the drugs of the drug panel.

The illustrative method of any of the proceeding illustrative embodiments wherein determining a subset of drugs of the drug panel comprises allowing a user to select a subset of drugs of the drug panel.

The illustrative method of any of the proceeding illustrative embodiments wherein determining initial RDDC combinations for use with the subset of drugs of the drug panel comprises selecting drug concentrations and radiation doses to apply to the cell lines.

The illustrative method of any of the proceeding illustrative embodiments wherein the drug concentrations comprise at least five drug concentrations.

The illustrative method of any of the proceeding illustrative embodiments wherein the radiation doses comprise at least 0 Gy and 2 Gy.

The illustrative method of any of the proceeding illustrative embodiments wherein determining full concentration-response relationships for the cell lines of the cell line panel using the initial RDDC combinations comprises, for each drug concentration of each drug of the subset of drugs and for each radiation dose to be examined: creating a duplicate cell line panel of the cell line panel; exposing the duplicate cell line panel to the drug concentration and the radiation dose; and measuring a cell count of each cell line of the duplicate cell line panel following exposure to the drug concentration and radiation dose.

The illustrative method of any of the proceeding illustrative embodiments wherein determining an RDDC combination subset from the initial RDDC combinations comprises: for each drug of the subset of drugs, creating full concentration-response curves for the drug with and without radiation and computing a change in area under curve (AUC) between the full concentration-response curves for the drug with and without radiation; and employing change in AUC for each drug of the subset of drugs to determine one or more drug concentrations for the RDDC combination subset.

The illustrative method of any of the proceeding illustrative embodiments wherein determining an RDDC combination subset from the initial RDDC combinations comprises: determining an initial set of drug concentrations; determining how the cell lines respond to the initial set of drug concentrations and a range of radiation doses; employing correlation analysis to determine a subset of drug concentrations from the initial set of drug concentrations based on how the cell lines respond to the initial set of drug concentrations and the range of radiation doses; and determining the RDDC combination subset based on the subset of drug concentrations.

The illustrative method of any of the proceeding illustrative embodiments wherein the range of radiation doses includes at least radiation doses of 0 Gy and 2 Gy or more.

The illustrative method of any of the proceeding illustrative embodiments wherein determining an initial set of drug concentrations comprises determining 5 or more drug concentrations.

The illustrative method of any of the proceeding illustrative embodiments wherein the RDDC combination subset comprises 3 or fewer drug concentrations for a panel of 100 or more drugs.

The illustrative method of any of the proceeding illustrative embodiments wherein the RDDC combination subset comprises one drug concentration for a panel of 100 or more drugs.

The illustrative method of any of the proceeding illustrative embodiments wherein screening the cell lines of the cell line panel using the RDDC combination subset for each drug within the drug panel comprises, for each drug of the drug panel and for each drug concentration and each radiation dose of the RDDC combination subset: creating a duplicate cell line panel of the cell line panel; exposing the duplicate cell line panel to the drug concentration and the radiation dose; and measuring a cell count of each cell line of the duplicate cell line panel following exposure to the drug concentration and radiation dose.

An illustrative method of screening drug-radiation interactions, comprising: determining a drug panel having a plurality of drugs to analyze; obtaining a cell line panel having one or more molecular parameters of interest and a plurality of cell lines; obtaining a cell line density and intrinsic radiotherapy response for each cell line in the cell line panel; determining a subset of drugs of the drug panel; determining initial radiation-dose-drug-concentration (RDDC) combinations for use with the subset of drugs of the drug panel; employing correlation analysis to determine a single drug concentration from the initial RDDC combinations based on how the cell lines respond to the initial RDDC combinations; and screening the cell lines of the cell line panel using the single drug concentration for each drug within the drug panel.

The illustrative method of any of the proceeding illustrative embodiments wherein employing correlation analysis to determine a single drug concentration from the initial RDDC combinations based on how the cell lines respond to the initial RDDC combinations comprises: for each drug of the subset of drugs, creating full concentration-response curves for the drug with and without radiation and computing a change in area under curve (AUC) between the full concentration-response curves for the drug with and without radiation; and employing correlation analysis and change in AUC for each drug of the subset of drugs to determine the single drug concentration.

An illustrative system for screening drug-radiation interactions, comprising: a dispenser system configured to dispense cells into cell lines of a cell line panel and drug concentrations into cell lines of the cell line panel; a radiation device configured to deliver radiation doses to the cell lines of the cell line panel; an incubator configured to incubate cell lines of the cell line panel; a measurement device configured to analyze the cell lines of the cell line panel; a processor coupled to the measurement device; and a memory coupled to the processor and having computer program instructions that, when executed by the processor, cause the processor to: obtain a list of drugs in a drug panel to analyze; obtain information related to a cell line panel having one or more molecular parameters of interest and a plurality of cell lines; obtain information related to a cell line density and intrinsic radiotherapy response for each cell line in the cell line panel; determine a subset of drugs of the drug panel; determine initial radiation-dose-drug-concentration (RDDC) combinations for use with the subset of drugs of the drug panel; determine full concentration-response relationships for the cell lines of the cell line panel using the initial RDDC combinations; determine an RDDC combination subset from the initial RDDC combinations based on the full concentration-response relationships; and screen the cell lines of the cell line panel using the RDDC combination subset for each drug within the drug panel.

The illustrative system of any of the proceeding illustrative embodiments wherein the memory includes computer program instructions that, when executed by the processor, cause the processor to obtain information related to the cell line panel from a database.

The illustrative system of any of the proceeding illustrative embodiments wherein the memory includes computer program instructions that, when executed by the processor, cause the processor to measure the cell line density of each cell line of the cell line panel by: employing the dispenser system to plate cells across a plurality of cell densities; employing the incubator to incubate the plated cells; employing the measurement device to identify densities that facilitate exponential growth rates; and identifying a timepoint at which cell line growth rate saturates.

The illustrative system of any of the proceeding illustrative embodiments wherein the memory includes computer program instructions that, when executed by the processor, cause the processor to measure the intrinsic radiotherapy response of each cell line of the cell line panel by: employing the dispenser system to create a duplicate cell line panel of the cell line panel; employing the radiation device to expose the duplicate cell line panel to a predetermined radiation dose; employing the incubator to incubate the duplicate cell line panel; and employing the measurement device to measure the cell count of each cell line of the duplicate cell line panel to determine the intrinsic radiotherapy response of the cell lines of the duplicate cell line panel.

The illustrative system of any of the proceeding illustrative embodiments wherein the memory includes computer program instructions that, when executed by the processor, cause the processor to determine full concentration-response relationships for the cell lines of the cell line panel using the initial RDDC combinations by, for each drug concentration of each drug of the subset of drugs and for each radiation dose to be examined: employing the dispenser system to create a duplicate cell line panel of the cell line panel; employing the dispenser system to expose the duplicate cell line panel to the drug concentration; employing the radiation device to expose the duplicate cell line panel to the radiation dose; and employing the measurement device to measure a cell count of each cell line of the duplicate cell line panel following exposure to the drug concentration and radiation dose.

The illustrative system of any of the proceeding illustrative embodiments wherein the memory includes computer program instructions that, when executed by the processor, cause the processor to determine an RDDC combination subset from the initial RDDC combinations by: for each drug of the subset of drugs, creating full concentration-response curves for the drug with and without radiation and computing a change in area under curve (AUC) between the full concentration-response curves for the drug with and without radiation; and employing change in AUC for each drug of the subset of drugs to determine one or more drug concentrations for the RDDC combination subset.

The illustrative system of any of the proceeding illustrative embodiments wherein the dispenser system includes a bulk dispenser and a fine dispenser.

The illustrative system of any of the proceeding illustrative embodiments wherein the radiation device is configured to deliver a radiation dose using at least one of x-rays, electrons, photons, and protons employing at least one of standard dosing, spatially fractionated radiation therapy, stereotactic body radiation therapy, and ultra-high-dose-rate radiotherapy.

The illustrative system of any of the proceeding illustrative embodiments wherein the radiation device is configured to deliver radiation doses including at least radiation doses of 0 Gy and 2 Gy or more.

The illustrative system of any of the proceeding illustrative embodiments wherein the measurement device is configured to measure a number of cells in each cell line of the cell line panel.

The illustrative system of any of the proceeding illustrative embodiments wherein the memory includes computer program instructions that, when executed by the processor, cause the processor to: for each drug within the drug panel and each drug concentration within the RDDC combination subset, employ the measurement device to measure cell count within each cell line with and without radiation; compare Z-score of change in cell count with and without radiation for each drug; and identify each drug having a Z-score of change in cell count below a predetermined threshold.

An illustrative system for screening drug-radiation interactions, comprising: a dispenser system configured to dispense cells into cell lines of a cell line panel and drug concentrations into cell lines of the cell line panel; a radiation device configured to deliver radiation doses to the cell lines of the cell line panel; an incubator configured to incubate cell lines of the cell line panel; a measurement device configured to analyze the cell lines of the cell line panel; a processor coupled to the measurement device; and a memory coupled to the processor and having computer program instructions that, when executed by the processor, cause the processor to: obtain a list of drugs in a drug panel to analyze; obtain information related to a cell line panel having one or more molecular parameters of interest and a plurality of cell lines; obtain information related to a cell line density and intrinsic radiotherapy response for each cell line in the cell line panel; determine a subset of drugs of the drug panel; determine initial radiation-dose-drug-concentration (RDDC) combinations for use with the subset of drugs of the drug panel; employ correlation analysis to determine a single drug concentration from the initial RDDC combinations based on how the cell lines respond to the initial RDDC combinations; and screen the cell lines of the cell line panel using the single drug concentration for each drug within the drug panel.

The illustrative system of any of the proceeding illustrative embodiments wherein the memory includes computer program instructions that, when executed by the processor, cause the processor to employ correlation analysis to determine a single drug concentration from the initial RDDC combinations based on how the cell lines respond to the initial RDDC combinations by: for each drug of the subset of drugs, creating full concentration-response curves for the drug with and without radiation and computing a change in area under curve (AUC) between the full concentration-response curves for the drug with and without radiation; and employing correlation analysis and change in AUC for each drug of the subset of drugs to determine the single drug concentration.

The illustrative system of any of the proceeding illustrative embodiments wherein the memory includes computer program instructions that, when executed by the processor, cause the processor to: for each drug within the drug panel and the single drug concentration, employ the measurement device to measure cell count within each cell line with and without radiation; compare Z-score of change in cell count with and without radiation for each drug; and identify drugs having a Z-score of change in cell count below a predetermined threshold.

The foregoing description discloses only example embodiments of the invention. Modifications of the above disclosed apparatus and methods which fall within the scope of the invention will be readily apparent to those of ordinary skill in the art.

Accordingly, while the present invention has been disclosed in connection with example embodiments thereof, it should be understood that other embodiments may fall within the spirit and scope of the invention, as defined by the following claims.

The foregoing disclosure provides illustration and description but is not intended to be exhaustive or to limit the implementations to the precise form disclosed. Modifications may be made in light of the above disclosure or may be acquired from practice of the implementations. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware may be used to implement the systems and/or methods based on the description herein.

Although particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

What is claimed is:

1. A method of screening drug-radiation interactions, comprising:
determining a drug panel having a plurality of drugs to analyze;
obtaining a cell line panel having one or more molecular parameters of interest and a plurality of cell lines;
obtaining a cell line density and intrinsic radiotherapy response for each cell line in the cell line panel;
determining a subset of drugs of the drug panel;
determining initial radiation-dose-drug-concentration (RDDC) combinations for use with the subset of drugs of the drug panel;
determining full concentration-response relationships for the cell lines of the cell line panel using the initial RDDC combinations;
determining an RDDC combination subset from the initial RDDC combinations based on the full concentration-response relationships; and
screening the cell lines of the cell line panel using the RDDC combination subset for each drug within the drug panel.

2. The method of claim 1 wherein obtaining a cell line panel having one or more molecular parameters of interest comprises obtaining cell lines having at least one of a genetic mutation, copy number variations, and a gene expression to be examined.

3. The method of claim 1 wherein the cell line panel comprises genetic heterogenicity for a cancer subtype.

4. The method of claim 1 wherein obtaining a cell line density for each cell line in the cell line panel includes at least one of determining growth kinetics and growth conditions for the cell line.

5. The method of claim 4 wherein determining a cell line density for each cell line comprises:
plating cells across a plurality of cell densities;
identifying densities that facilitate exponential growth rates; and
identifying a timepoint at which cell line growth rate saturates.

6. The method of claim 1 wherein determining a subset of drugs of the drug panel comprises selecting 10% or fewer of the drugs of the drug panel.

7. The method of claim 1 wherein determining a subset of drugs of the drug panel comprises selecting 5% or fewer of the drugs of the drug panel.

8. The method of claim 1 wherein determining a subset of drugs of the drug panel comprises allowing a user to select a subset of drugs of the drug panel.

9. The method of claim 1 wherein determining initial RDDC combinations for use with the subset of drugs of the drug panel comprises selecting drug concentrations and radiation doses to apply to the cell lines.

10. The method of claim 9 wherein the drug concentrations comprise at least five drug concentrations.

11. The method of claim 9 wherein the radiation doses comprise at least 0 Gy and 2 Gy.

12. The method of claim 1 wherein determining full concentration-response relationships for the cell lines of the cell line panel using the initial RDDC combinations comprises, for each drug concentration of each drug of the subset of drugs and for each radiation dose to be examined:
creating a duplicate cell line panel of the cell line panel;
exposing the duplicate cell line panel to the drug concentration and the radiation dose; and
measuring a cell count of each cell line of the duplicate cell line panel following exposure to the drug concentration and radiation dose.

13. The method of claim 1 wherein determining an RDDC combination subset from the initial RDDC combinations comprises:
for each drug of the subset of drugs, creating full concentration-response curves for the drug with and without radiation and computing a change in area under curve (AUC) between the full concentration-response curves for the drug with and without radiation; and
employing change in AUC for each drug of the subset of drugs to determine one or more drug concentrations for the RDDC combination subset.

14. The method of claim 1 wherein determining an RDDC combination subset from the initial RDDC combinations comprises:
determining an initial set of drug concentrations;
determining how the cell lines respond to the initial set of drug concentrations and a range of radiation doses;
employing correlation analysis to determine a subset of drug concentrations from the initial set of drug concentrations based on how the cell lines respond to the initial set of drug concentrations and the range of radiation doses; and
determining the RDDC combination subset based on the subset of drug concentrations.

15. The method of claim 14 wherein the range of radiation doses includes at least radiation doses of 0 Gy and 2 Gy or more.

16. The method of claim 14 wherein determining an initial set of drug concentrations comprises determining 5 or more drug concentrations.

17. The method of claim 1 wherein the RDDC combination subset comprises 3 or fewer drug concentrations for a panel of 100 or more drugs.

18. The method of claim 17 wherein the RDDC combination subset comprises one drug concentration for a panel of 100 or more drugs.

19. The method of claim 17 wherein screening the cell lines of the cell line panel using the RDDC combination subset for each drug within the drug panel comprises, for each drug of the drug panel and for each drug concentration and each radiation dose of the RDDC combination subset:
creating a duplicate cell line panel of the cell line panel;
exposing the duplicate cell line panel to the drug concentration and the radiation dose; and
measuring a cell count of each cell line of the duplicate cell line panel following exposure to the drug concentration and radiation dose.

20. A method of screening drug-radiation interactions, comprising:
determining a drug panel having a plurality of drugs to analyze;
obtaining a cell line panel having one or more molecular parameters of interest and a plurality of cell lines;
obtaining a cell line density and intrinsic radiotherapy response for each cell line in the cell line panel;
determining a subset of drugs of the drug panel;
determining initial radiation-dose-drug-concentration (RDDC) combinations for use with the subset of drugs of the drug panel;
employing correlation analysis to determine a single drug concentration from the initial RDDC combinations based on how the cell lines respond to the initial RDDC combinations; and
screening the cell lines of the cell line panel using the single drug concentration for each drug within the drug panel.

21. The method of claim 20 wherein employing correlation analysis to determine a single drug concentration from the initial RDDC combinations based on how the cell lines respond to the initial RDDC combinations comprises:
for each drug of the subset of drugs, creating full concentration-response curves for the drug with and without radiation and computing a change in area under curve (AUC) between the full concentration-response curves for the drug with and without radiation; and
employing correlation analysis and change in AUC for each drug of the subset of drugs to determine the single drug concentration.

22. A system for screening drug-radiation interactions, comprising:
a dispenser system configured to dispense cells into cell lines of a cell line panel and drug concentrations into cell lines of the cell line panel;
a radiation device configured to deliver radiation doses to the cell lines of the cell line panel;
an incubator configured to incubate cell lines of the cell line panel;
a measurement device configured to analyze the cell lines of the cell line panel;
a processor coupled to the measurement device; and
a memory coupled to the processor and having computer program instructions that, when executed by the processor, cause the processor to:
obtain a list of drugs in a drug panel to analyze;
obtain information related to a cell line panel having one or more molecular parameters of interest and a plurality of cell lines;
obtain information related to a cell line density and intrinsic radiotherapy response for each cell line in the cell line panel;
determine a subset of drugs of the drug panel;
determine initial radiation-dose-drug-concentration (RDDC) combinations for use with the subset of drugs of the drug panel;
determine full concentration-response relationships for the cell lines of the cell line panel using the initial RDDC combinations;
determine an RDDC combination subset from the initial RDDC combinations based on the full concentration-response relationships; and
screen the cell lines of the cell line panel using the RDDC combination subset for each drug within the drug panel.

23. The system of claim 22 wherein the memory includes computer program instructions that, when executed by the processor, cause the processor to obtain information related to the cell line panel from a database.

24. The system of claim 22 wherein the memory includes computer program instructions that, when executed by the processor, cause the processor to measure the cell line density of each cell line of the cell line panel by:
employing the dispenser system to plate cells across a plurality of cell densities;
employing the incubator to incubate the plated cells;
employing the measurement device to identify densities that facilitate exponential growth rates; and
identifying a timepoint at which cell line growth rate saturates.

25. The system of claim 22 wherein the memory includes computer program instructions that, when executed by the processor, cause the processor to measure the intrinsic radiotherapy response of each cell line of the cell line panel by:
employing the dispenser system to create a duplicate cell line panel of the cell line panel;
employing the radiation device to expose the duplicate cell line panel to a predetermined radiation dose;
employing the incubator to incubate the duplicate cell line panel; and
employing the measurement device to measure the cell count of each cell line of the duplicate cell line panel to determine the intrinsic radiotherapy response of the cell lines of the duplicate cell line panel.

26. The system of claim 22 wherein the memory includes computer program instructions that, when executed by the processor, cause the processor to determine full concentration-response relationships for the cell lines of the cell line panel using the initial RDDC combinations by, for each drug concentration of each drug of the subset of drugs and for each radiation dose to be examined:
employing the dispenser system to create a duplicate cell line panel of the cell line panel;
employing the dispenser system to expose the duplicate cell line panel to the drug concentration;
employing the radiation device to expose the duplicate cell line panel to the radiation dose; and
employing the measurement device to measure a cell count of each cell line of the duplicate cell line panel following exposure to the drug concentration and radiation dose.

27. The system of claim 22 wherein the memory includes computer program instructions that, when executed by the processor, cause the processor to determine an RDDC combination subset from the initial RDDC combinations by:
for each drug of the subset of drugs, creating full concentration-response curves for the drug with and without radiation and computing a change in area under curve (AUC) between the full concentration-response curves for the drug with and without radiation; and
employing change in AUC for each drug of the subset of drugs to determine one or more drug concentrations for the RDDC combination subset.

28. The system of claim 22 wherein the dispenser system includes a bulk dispenser and a fine dispenser.

29. The system of claim 22 wherein the radiation device is configured to deliver a radiation dose using at least one of x-rays, electrons, photons, and protons employing at least one of standard dosing, spatially fractionated radiation therapy, stereotactic body radiation therapy, and ultra-high-dose-rate radiotherapy.

30. The system of claim 22 wherein the radiation device is configured to deliver radiation doses including at least radiation doses of 0 Gy and 2 Gy or more.

31. The system of claim 22 wherein the measurement device is configured to measure a number of cells in each cell line of the cell line panel.

32. The system of claim 22 wherein the memory includes computer program instructions that, when executed by the processor, cause the processor to:
for each drug within the drug panel and each drug concentration within the RDDC combination subset, employ the measurement device to measure cell count within each cell line with and without radiation;
compare Z-score of change in cell count with and without radiation for each drug; and
identify each drug having a Z-score of change in cell count below a predetermined threshold.

33. A system for screening drug-radiation interactions, comprising:
a dispenser system configured to dispense cells into cell lines of a cell line panel and drug concentrations into cell lines of the cell line panel;
a radiation device configured to deliver radiation doses to the cell lines of the cell line panel;
an incubator configured to incubate cell lines of the cell line panel;
a measurement device configured to analyze the cell lines of the cell line panel;
a processor coupled to the measurement device; and
a memory coupled to the processor and having computer program instructions that, when executed by the processor, cause the processor to:
obtain a list of drugs in a drug panel to analyze;
obtain information related to a cell line panel having one or more molecular parameters of interest and a plurality of cell lines;
obtain information related to a cell line density and intrinsic radiotherapy response for each cell line in the cell line panel;
determine a subset of drugs of the drug panel;
determine initial radiation-dose-drug-concentration (RDDC) combinations for use with the subset of drugs of the drug panel;
employ correlation analysis to determine a single drug concentration from the initial RDDC combinations based on how the cell lines respond to the initial RDDC combinations; and
screen the cell lines of the cell line panel using the single drug concentration for each drug within the drug panel.

34. The system of claim 33 wherein the memory includes computer program instructions that, when executed by the processor, cause the processor to employ correlation analysis to determine a single drug concentration from the initial RDDC combinations based on how the cell lines respond to the initial RDDC combinations by:
for each drug of the subset of drugs, creating full concentration-response curves for the drug with and without radiation and computing a change in area under curve (AUC) between the full concentration-response curves for the drug with and without radiation; and
employing correlation analysis and change in AUC for each drug of the subset of drugs to determine the single drug concentration.

35. The system of claim 33 wherein the memory includes computer program instructions that, when executed by the processor, cause the processor to:
for each drug within the drug panel and the single drug concentration, employ the measurement device to measure cell count within each cell line with and without radiation;
compare Z-score of change in cell count with and without radiation for each drug; and
identify drugs having a Z-score of change in cell count below a predetermined threshold.
